(19) United States
(12) Patent Application Publication
(10) Pub. No.: US 2023/0335246 A1
(43) Pub. Date: Oct. 19, 2023

(54) System, Method, and Computer Program Product for Validating Prescriptions

(71) Applicant: Andrew Siegfried, Oakdale, PA (US)

(72) Inventor: Andrew Siegfried, Oakdale, PA (US)

(21) Appl. No.: 18/300,960

(22) Filed: Apr. 14, 2023

Related U.S. Application Data
(60) Provisional application No. 63/332,019, filed on Apr. 18, 2022.

(51) Int. Cl. G16H 20/10; G16H 70/40; G16H 10/60

(52) U.S. Cl. CPC G16H 20/10; G16H 70/40; G16H 10/60

(57) ABSTRACT

A system, method, and computer program product for validating prescriptions may receive, from a pharmacy system, a prescription associated with a patient; receive, from a drug database system, an approved dosage and an approved condition; generate, using a machine learning model, a validation decision associated with the prescription; and provide, to the pharmacy system, the validation decision. The validation decision may cause the pharmacy system to one of (i) automatically validate the prescription, in response to the validation decision including an indication that the prescription is valid and (ii) automatically lock processing of the prescription such that the pharmacy system is prevented from filling the prescription until manual input associated with the prescription is received from a pharmacist, in response to the validation decision including an indication that the prescription is invalid.

### CROSS-REFERENCE TO RELATED APPLICATION

This application claims priority to U.S. Provisional Patent Application No. 63/332,019 filed on Apr. 18, 2022, the disclosure of which is hereby incorporated by reference in its entirety.

### BACKGROUND

### 1. Field

This disclosure relates to pharmacy platforms and, in some non-limiting embodiments or aspects, to systems, methods, and computer program products for validating prescriptions using machine learning.

### 2. Technical Considerations

Pharmacies today are continually being asked to do more with less; gone are the days of pharmacist overlap and extra technician hours. These common trends across the pharmacy landscape are leading to unnecessary errors and are prohibiting pharmacists from being true clinicians. Labor is the most expensive variable within cost to fill models, and with ever shrinking margins, most pharmacies are left with no choice but to cut labor and attempt to increase revenue streams with additional services, which unfortunately leaves the pharmacist doing more with less.

For example, retail chains have turned into massive dispensing factories, operating on razor thin margins and expecting the pharmacist to be perfect, catching mistakes from the physician, the nurse, and the pharmacy tech, all while working in a high stress environment with less support staff and competing priorities. The patient is the one who ultimately suffers from this type of pharmacy. The pharmacist is supposed to be the last line of defense protecting and empowering the patient to take their medication correctly, avoid side effects, while understanding drug interactions with the idealized outcome of successfully treating their underlying condition whether acute or chronic. Unfortunately, this environment inhibits the pharmacist from having meaningful clinical conversations with their patients and thereby hinders them from practicing at the top of their license. This model is ultimately taking away the true value pharmacists can provide within the healthcare ecosystem and unfortunately mistakes happen. Most of these mistakes are obvious and should have been caught, but due to the current environment, they are missed. For example, errors occur at a rate of 4 per day in a pharmacy filling 250 prescriptions daily, 51.5 million errors out of the 3 billion prescriptions dispensed annually. Counseling interactions and interventions are also missed; patients do not want to ask their questions due to the lack of time and the chaos of the retail pharmacy.

### SUMMARY

Accordingly, provided are improved systems, devices, products, apparatus, and/or methods for validating prescriptions.

Non-limiting embodiments or aspects are set forth in the following numbered clauses:

Clause 1. A system, comprising: at least one processor programmed and/or configured to: receive, from a pharmacy system, a prescription associated with a patient, wherein the prescription includes a drug code associated with a drug, a diagnosis code associated with a condition associated with the patient, a prescribed dosage associated with the drug, and a prescribed quantity associated with the drug; provide, to a drug database system, the drug code and the diagnosis code; in response to providing the drug code and the diagnosis code to the drug database system, receive, from the drug database system, an approved dosage and an approved condition; generate, using a machine learning model, a validation decision associated with the prescription by providing as input to the machine learning model the drug code, the diagnosis code, the prescribed dosage, the prescribed quantity, the approved dosage, and the approved condition, and receiving as output from the machine learning model the validation decision, wherein the validation decision includes an indication that the prescription is one of valid and invalid; and provide, to the pharmacy system, the validation decision, wherein the validation decision causes the pharmacy system to one of (i) automatically validate the prescription, in response to the validation decision including the indication that the prescription is valid and (ii) automatically lock processing of the prescription such that the pharmacy system is prevented from filling the prescription until manual input associated with the prescription is received from a pharmacist, in response to the validation decision including the indication that the prescription is invalid.

Clause 2. The system of clause 1, wherein the validation decision includes the indication that the prescription is invalid that causes the pharmacy system to automatically lock processing of the prescription such that the pharmacy system is prevented from filling the prescription until the manual input associated with the prescription is received from the pharmacist, and wherein the at least one processor is further programmed and/or configured to: receive, from the pharmacy system, an outcome associated with the prescription; and update, based on the outcome associated with the prescription, the machine learning model.

Clause 3. The system of clauses 1 or 2, wherein the diagnosis code is associated with a different condition than the approved condition.

Clause 4. The system of any of clauses 1-3, wherein the outcome associated with the prescription includes an indication that the manual input from the pharmacist validated the prescription by overriding the validation decision including the indication that the prescription is invalid.

Clause 5. The system of any of clauses 1-4, wherein the prescription includes an electronic prescription, and wherein the electronic prescription is generated by a prescriber system.

Clause 6. The system of any of clauses 1-5, wherein the at least one processor is further programmed and/or configured to: obtain patient data associated with the patient, wherein the patient data includes at least one of the following parameters: a patient name, a patient date of birth or age, a patient sex, a patient weight, a patient health condition, a patient allergy, a patient medication, a contraindication, a comorbidity, or any combination thereof, wherein the at least one processor generates the validation decision associated with the prescription by further providing as input to the machine learning model at least one parameter of the patient data.

Clause 7. The system of any of clauses 1-6, wherein the approved dosage is dependent on a weight of the patient, and wherein the at least one processor generates the validation decision associated with the prescription by further providing as input to the machine learning model the patient weight.

Clause 8. A computer-implemented method, comprising: receiving, with at least one processor, from a pharmacy system, a prescription associated with a patient, wherein the prescription includes a drug code associated with a drug, a diagnosis code associated with a condition associated with the patient, a prescribed dosage associated with the drug, and a prescribed quantity associated with the drug; providing, with the at least one processor, to a drug database system, the drug code and the diagnosis code; in response to providing the drug code and the diagnosis code to the drug database system, receiving, with the at least one processor, from the drug database system, an approved dosage and an approved condition; generating, with the at least one processor, using a machine learning model, a validation decision associated with the prescription by providing as input to the machine learning model the drug code, the diagnosis code, the prescribed dosage, the prescribed quantity, the approved dosage, and the approved condition, and receiving as output from the machine learning model the validation decision, wherein the validation decision includes an indication that the prescription is one of valid and invalid; and providing, with the at least one processor, to the pharmacy system, the validation decision, wherein the validation decision causes the pharmacy system to one of (i) automatically validate the prescription, in response to the validation decision including the indication that the prescription is valid and (ii) automatically lock processing of the prescription such that the pharmacy system is prevented from filling the prescription until manual input associated with the prescription is received from a pharmacist, in response to the validation decision including the indication that the prescription is invalid.

Clause 9. The computer-implemented method of clause 8, wherein the validation decision includes the indication that the prescription is invalid that causes the pharmacy system to automatically lock processing of the prescription such that the pharmacy system is prevented from filling the prescription until the manual input associated with the prescription is received from the pharmacist, and wherein the method further comprises: receiving, with the at least one processor, from the pharmacy system, an outcome associated with the prescription; and updating, with the at least one processor, based on the outcome associated with the prescription, the machine learning model.

Clause 10. The computer-implemented method of clauses 8 or 9, wherein the diagnosis code is associated with a different condition than the approved condition.

Clause 11. The computer-implemented method of any of clauses 8-10, wherein the outcome associated with the prescription includes an indication that the manual input from the pharmacist validated the prescription by overriding the validation decision including the indication that the prescription is invalid.

Clause 12. The computer-implemented method of any of clauses 8-11, wherein the prescription includes an electronic prescription, and wherein the electronic prescription is generated by a prescriber system.

Clause 13. The computer-implemented method of any of clauses 8-12, further comprising: obtaining, with the at least one processor, patient data associated with the patient, wherein the patient data includes at least one of the following parameters: a patient name, a patient date of birth or age, a patient sex, a patient weight, a patient health condition, a patient allergy, a patient medication, a contraindication, a comorbidity, or any combination thereof, wherein the at least one processor generates the validation decision associated with the prescription by further providing as input to the machine learning model at least one parameter of the patient data.

Clause 14. The computer-implemented method of any of clauses 8-13, wherein the approved dosage is dependent on a weight of the patient, and wherein the at least one processor generates the validation decision associated with the prescription by further providing as input to the machine learning model the patient weight.

Clause 15. A computer program product comprising at least one non-transitory computer-readable medium including program instructions that, when executed by at least one processor, cause the at least one processor to: receive, from a pharmacy system, a prescription associated with a patient, wherein the prescription includes a drug code associated with a drug, a diagnosis code associated with a condition associated with the patient, a prescribed dosage associated with the drug, and a prescribed quantity associated with the drug; provide, to a drug database system, the drug code and the diagnosis code; in response to providing the drug code and the diagnosis code to the drug database system, receive, from the drug database system, an approved dosage and an approved condition; generate, using a machine learning model, a validation decision associated with the prescription by providing as input to the machine learning model the drug code, the diagnosis code, the prescribed dosage, the prescribed quantity, the approved dosage, and the approved condition, and receiving as output from the machine learning model the validation decision, wherein the validation decision includes an indication that the prescription is one of valid and invalid; and provide, to the pharmacy system, the validation decision, wherein the validation decision causes the pharmacy system to one of (i) automatically validate the prescription, in response to the validation decision including the indication that the prescription is valid and (ii) automatically lock processing of the prescription such that the pharmacy system is prevented from filling the prescription until manual input associated with the prescription is received from a pharmacist, in response to the validation decision including the indication that the prescription is invalid.

Clause 16. The computer program product of clause 15, wherein the validation decision includes the indication that the prescription is invalid that causes the pharmacy system to automatically lock processing of the prescription such that the pharmacy system is prevented from filling the prescription until the manual input associated with the prescription is received from the pharmacist, and wherein the program instructions, when executed by the at least one processor, further cause the at least one processor to: receive, from the pharmacy system, an outcome associated with the prescription; and update, based on the outcome associated with the prescription, the machine learning model.

Clause 17. The computer program product of clauses 15 or 16, wherein the diagnosis code is associated with a different condition than the approved condition.

Clause 18. The computer program product of any of clauses 15-17, wherein the outcome associated with the prescription includes an indication that the manual input from the pharmacist validated the prescription by overriding the validation decision including the indication that the prescription is invalid.

Clause 19. The computer program product of any of clauses 15-18, wherein the prescription includes an electronic prescription, and wherein the electronic prescription is generated by a prescriber system.

Clause 20. The computer program product of any of clauses 15-19, wherein the program instructions, when executed by the at least one processor, further cause the at least one processor to: obtain patient data associated with the patient, wherein the patient data includes at least one of the following parameters: a patient name, a patient date of birth or age, a patient sex, a patient weight, a patient health condition, a patient allergy, a patient medication, a contraindication, a comorbidity, or any combination thereof, wherein the at least one processor generates the validation decision associated with the prescription by further providing as input to the machine learning model at least one parameter of the patient data, wherein the approved dosage is dependent on a weight of the patient, and wherein the at least one processor generates the validation decision associated with the prescription by further providing as input to the machine learning model the patient weight.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of non-limiting embodiments or aspects of an environment in which systems, devices, products, apparatus, and/or methods, described herein, may be implemented;

FIG. 2 is a diagram of non-limiting embodiments or aspects of components of one or more devices and/or one or more systems of FIG. 1;

FIG. 3 is a flowchart of non-limiting embodiments or aspects of a process for validating prescriptions; and

FIG. 4 is a diagram of an implementation of non-limiting embodiments or aspects of a process for validating prescriptions.

### DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature.

Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

As used herein, the term “mobile device” may refer to one or more portable electronic devices configured to communicate with one or more networks. As an example, a mobile device may include a cellular phone (e.g., a smartphone or standard cellular phone), a portable computer (e.g., a tablet computer, a laptop computer, etc.), a wearable device (e.g., a watch, pair of glasses, lens, clothing, and/or the like), a personal digital assistant (PDA), and/or other like devices. The terms “client device” and “user device,” as used herein, refer to any electronic device that is configured to communicate with one or more servers or remote devices and/or systems. A client device or user device may include a mobile device, a network-enabled appliance (e.g., a network-enabled television, refrigerator, thermostat, and/or the like), a computer, a POS system, and/or any other device or system capable of communicating with a network.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a PDA, and/or other like devices. A computing device may also be a desktop computer or other form of non-mobile computer.

As used herein, the term “server” and/or “processor” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, POS devices, mobile devices, etc.) directly or indirectly communicating in the network environment may constitute a “system.” Reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

As used herein, the term “application programming interface” (API) may refer to computer code that allows communication between different systems or (hardware and/or software) components of systems. For example, an API may include function calls, functions, subroutines, communication protocols, fields, and/or the like usable and/or accessible by other systems or other (hardware and/or software) components of systems.

As used herein, the term “user interface” or “graphical user interface” refers to a generated display, such as one or more graphical user interfaces (GUIs) with which a user may interact, either directly or indirectly (e.g., through a keyboard, mouse, touchscreen, etc.).

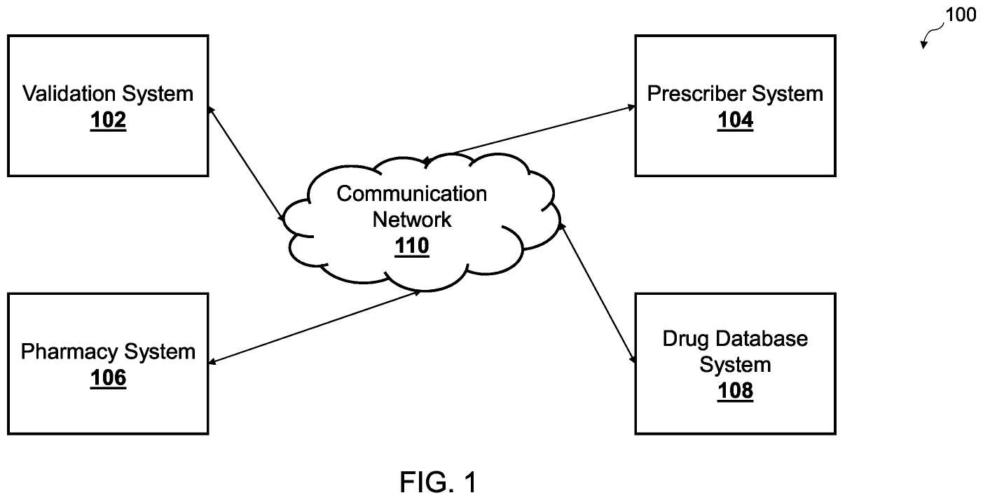

Referring now to FIG. 1, FIG. 1 is a diagram of an example environment 100 in which devices, systems, methods, and/or products described herein, may be implemented. As shown in FIG. 1, environment 100 includes validation system 102, prescriber system 104, pharmacy system 106, drug database system 108, and/or communication network 110. Validation system 102, prescriber system 104, pharmacy system 106, and/or drug database system 108 may interconnect (e.g., establish a connection to communicate, etc.) via wired connections, wireless connections, or a combination of wired and wireless connections.

Validation system 102 may include one or more devices capable of receiving information and/or data from prescriber system 104, pharmacy system 106, and/or drug database system 108 (e.g., via communication network 110, etc.) and/or communicating information and/or data to prescriber system 104, pharmacy system 106, and drug database system 108 (e.g., via communication network 110, etc.). For example, validation system 102 may include a computing device, such as a server, a group of servers, and/or other like devices.

Prescriber system 104 may include one or more devices capable of receiving information and/or data from validation system 102, pharmacy system 106, and/or drug database system 108 (e.g., via communication network 110, etc.) and/or communicating information and/or data to validation system 102, pharmacy system 106, and drug database system 108 (e.g., via communication network 110, etc.). For example, validation system 102 may include a computing device, such as a server, a group of servers, and/or other like devices. In some non-limiting embodiments or aspects, prescriber system 104 may be associated with a person and/or an entity including persons who prescribe medication, such as a physician, physician assistant, and/or nurse practitioner, and/or the like. In some non-limiting embodiments or aspects, prescriber system 104 may be configured to generate and provide electronic prescriptions through the electronic transmission of a prescription directly to a pharmacy through electronic health record (EHR) technology, or via a standalone electronic prescribing suite.

Pharmacy system 106 may include one or more devices capable of receiving information and/or data from validation system 102, prescriber system 104, and/or drug database system 108 (e.g., via communication network 110, etc.) and/or communicating information and/or data to validation system 102, prescriber system 104, and drug database system 108 (e.g., via communication network 110, etc.). For example, pharmacy system 106 may include a computing device, such as a server, a group of servers, and/or other like devices. In some non-limiting embodiments or aspects, prescriber system 104 may be associated with a pharmacy including one or more pharmacists authorized to fill prescriptions for patients.

Drug database system 108 may include one or more devices capable of receiving information and/or data from validation system 102, prescriber system 104, and/or pharmacy system 106 (e.g., via communication network 110, etc.) and/or communicating information and/or data to validation system 102, prescriber system 104, and pharmacy system 106 (e.g., via communication network 110, etc.). For example, drug database system 108 may include a computing device, such as a server, a group of servers, and/or other like devices. In some non-limiting embodiments or aspects, drug database system 108 may include one or more databases. For example, drug database system 108 may include a drug utilization review (DUR) system, such as Medi-Span®, and/or the like.

Communication network 110 may include one or more wired and/or wireless networks. For example, communication network 110 may include a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

The number and arrangement of devices and systems shown in FIG. 1 is provided as an example. There may be additional devices and/or systems, fewer devices and/or systems, different devices and/or systems, or differently arranged devices and/or systems than those shown in FIG. 1. Furthermore, two or more devices and/or systems shown in FIG. 1 may be implemented within a single device and/or system, or a single device and/or system shown in FIG. 1 may be implemented as multiple, distributed devices and/or systems. Additionally, or alternatively, a set of devices and/or systems (e.g., one or more devices or systems) of environment 100 may perform one or more functions described as being performed by another set of devices and/or systems of environment 100.

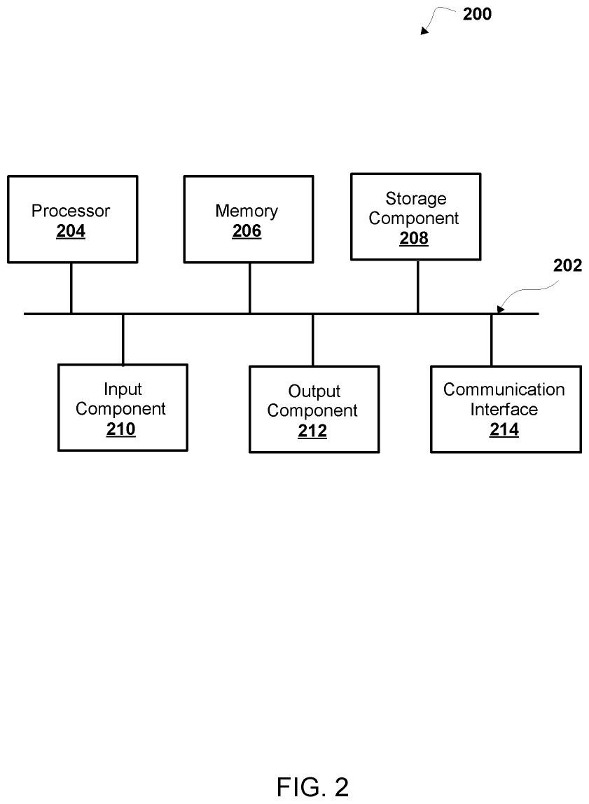

Referring now to FIG. 2, FIG. 2 is a diagram of example components of a device 200. Device 200 may correspond to one or more devices of validation system 102, one or more devices of prescriber system 104, one or more devices of pharmacy system 106, and/or one or more devices of drug database system 108. In some non-limiting embodiments or aspects, one or more devices of validation system 102, one or more devices of prescriber system 104, one or more devices of pharmacy system 106, and/or one or more devices of drug database system 108 may include at least one device 200 and/or at least one component of device 200. As shown in FIG. 2, device 200 may include a bus 202, a processor 204, memory 206, a storage component 208, an input component 210, an output component 212, and a communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 (e.g., a central processing unit (CPU), a graphics processing unit (GPU), etc.) executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

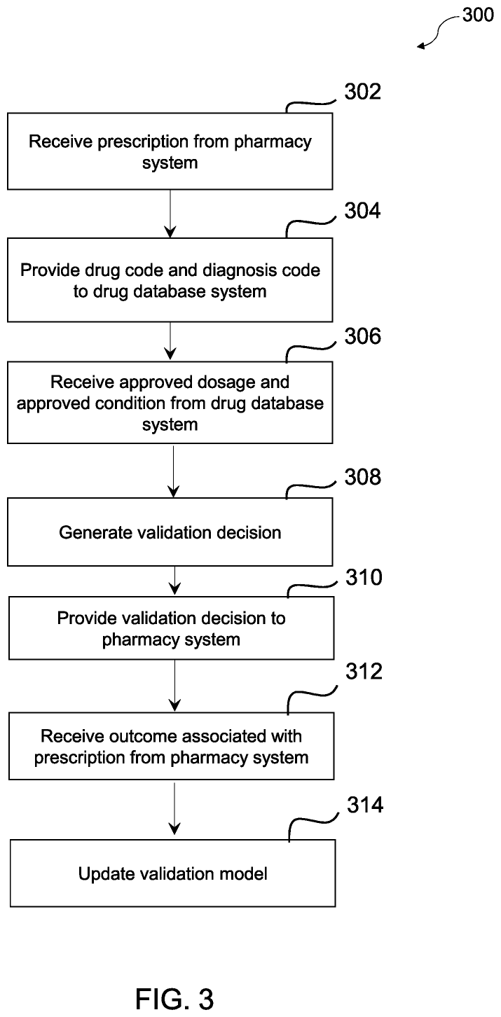

Referring now to FIG. 3, FIG. 3 is a flowchart of non-limiting embodiments or aspects of a process 300 for validating prescriptions. In some non-limiting embodiments or aspects, one or more of the steps of process 300 may be performed (e.g., completely, partially, etc.) by validation system 102 (e.g., one or more devices of validation system 102, etc.). In some non-limiting embodiments or aspects, one or more of the steps of process 300 may be performed (e.g., completely, partially, etc.) by another device or a group of devices separate from or including validation system 102, such as one or more devices of prescriber system 104, one or more devices of pharmacy system 106, and/or one or more devices of drug database system 108.

As shown in FIG. 3, at step 302, process 300 includes receiving a prescription from a pharmacy system. For example, validation system 102 may receive, from pharmacy system 106, a prescription associated with a patient. The prescription may include a drug code (e.g., a national drug code (NDC), etc.) associated with a drug, a diagnosis code (e.g., an International Classification of Diseases 10th Revision (ICD-10) code, etc.) associated with a condition associated with the patient, a prescribed dosage associated with the drug, and a prescribed quantity associated with the drug.

A condition associated with a patient may include a specific disease associated with the patient, such as diabetes, Hepatitis C, and/or the like. For example, a doctor or other prescriber may diagnose a patient with a condition.

A prescribed dosage associated with a drug may include a size or amount and/or a frequency of a dose of a medicine or drug (e.g., 50 milligrams of insulin each day, etc.). A prescribed quantity associated with a drug may include a number of unit doses of the drug. For example, a unit dose may include an amount of a medication administered to a patient in a single dose, and a prescribed quantity may include a number of unit doses prescribed by a prescription. In some non-limiting embodiments or aspects, a prescribed dosage may include further instructions from the prescriber indicating how the patient should use the drug or medication, such as a manner in which the drug should be taken (e.g., with food, as needed, etc.) and/or other instructions associated with taking the drug (e.g., avoid alcohol, avoid sun exposure, etc.).

In some non-limiting embodiments or aspects, validation system 102 obtains patient data associated with a patient. The patient data may include at least one of the following parameters: a patient name, a patient date of birth or age, a patient sex, a patient weight, a patient health condition, a patient allergy, a patient medication, a contraindication (e.g., a specific situation in which a drug, procedure, or surgery should not be used because it may be harmful to the person, etc.), a comorbidity (e.g., a simultaneous presence of two or more diseases or medical conditions in a patient, etc.), a patient lab value (e.g., a patient serum creatinine level including trough concentrations, a patient serum digoxin level, a patient liver function level, a patient prothrombin time test with an international normalized ratio (PT/INR) etc.), a patient pharmacogenetics factor (e.g., a thiopurine methyltransferase (TPMT) genotype, a CYP2D6 gene function, a CYP2C19 gene function, etc.), or any combination thereof. For example, patient data may be included in the prescription and/or retrieved by validation system 102 from pharmacy system 106 and/or another patient database via a separate query.

A pharmacogenetics factor may include variations in drug response due to genetic makeup. The activity of drug-metabolizing enzymes often varies widely among healthy people, making metabolism highly variable. Drug elimination rates vary up to 40-fold. Genetic factors and aging seem to account for most of these variations. Pharmacogenetics variation (e.g., in acetylation, hydrolysis, oxidation, or drug-metabolizing enzymes) can have clinical consequences. For example, if patients metabolize certain drugs rapidly, they may require higher, more frequent doses to achieve therapeutic concentrations; if patients metabolize certain drugs slowly, they may need lower, less frequent doses to avoid toxicity, particularly of drugs with a narrow margin of safety. For example, patients with inflammatory bowel disease who require Azathioprine therapy are now routinely tested for TPMT genotype to determine the most appropriate starting dose for drug therapy. As an example, in the use of Amitriptyline for depression, the elimination of the drug is linked to two genes CYP2D6 and CYP2C19, and dosing of the drug may be adjusted based on how these two genes function (e.g., a CYP2D6 metabolization rate, etc.) in the patient. Many genetic differences cannot be predicted before drug therapy, but for an increasing number of drugs (e.g., Carbamazepine, Clopidogrel, Warfarin, etc.), changes in effectiveness and risk of toxicity have been specifically associated with certain genetic variations. Also, many environmental and developmental factors can interact with each other and with genetic factors to affect drug response.

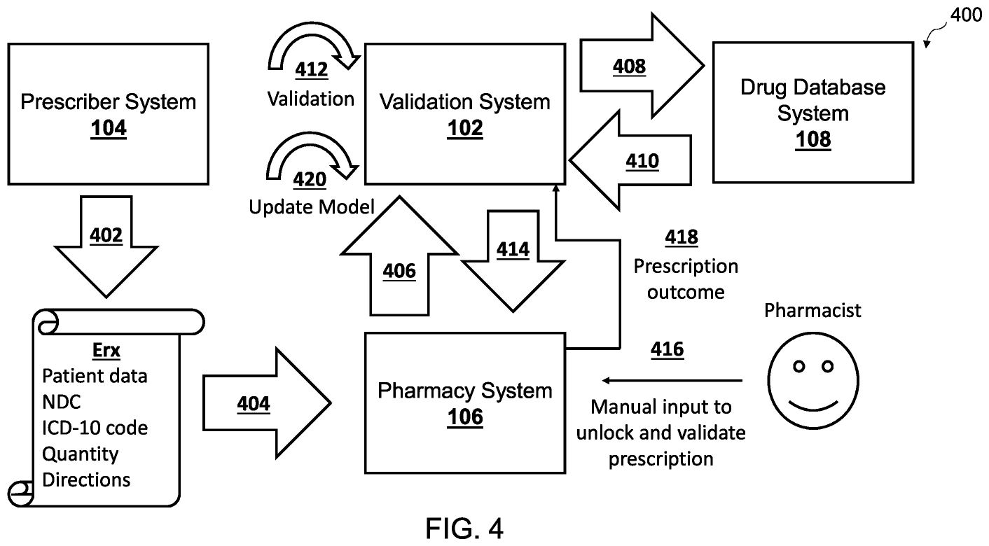

In some non-limiting embodiments or aspects, a prescription includes an electronic prescription. For example, and referring also to FIG. 4, which is diagram of an implementation 400 of non-limiting embodiments or aspects of a process for validating prescriptions, at reference number 402, prescriber system 104 may generate an electronic prescription ERx. As an example, a physician or nurse may enter the prescription into prescriber system 104 by selecting from a list of NDC numbers which are specific to the drug or medication being prescribed to enter the drug or medication, quantity, directions, refills, indication or diagnosis, and/or patient data. As shown at reference number 404, prescriber system 104 may electronically submit the electronic prescription ERx to pharmacy system 106, at which the electronic prescription is received and/or stored pre-populated in a pharmacy database. In such an example, validation system 102 may pull past patient history (e.g., contraindications associated with patient conditions and/or allergies, etc.) and/or other patient data from pharmacy system 106 and/or another system or database (e.g., a hospital database, an insurance database, prescriber system 104, etc.).

Conventionally, after a pharmacy technician confirms the electronic prescription ERx received from prescriber system 104, a pharmacist manually reviews and validates the prescription in pharmacy system 106. However, as shown at reference number 406, validation system 102 may instead use an API connection with a live Health Insurance Portability and Accountability Act (HIPAA) secure connection to pharmacy system 106 and/or the pharmacy database to automatically pull the electronic prescription ERx from pharmacy system 106 and/or the pharmacy database (e.g., in response to the electronic prescription ERx being stored in the pharmacy database, etc.). In such an example, validation system 102 may use multiple different API connections with multiple different pharmacy software systems to work with multiple different pharmacies/pharmacy software. For example, independent pharmacies, small grocers, and/or regional chains may use a third party pharmacy software, such as QS-1, PioneerRx, MicroMerchants, PDX, and/or the like, and validation system 102 may be pharmacy software agnostic by using custom APIs to access electronic prescriptions ERxs from the multiple different pharmacy software systems. As an example, larger retail or chain pharmacies may use proprietary pharmacy software, and validation system 102 may be pharmacy software agnostic by using custom APIs to access electronic prescriptions (ERxs) from these proprietary pharmacy software systems.

As shown in FIG. 3, at step 304, process 300 includes providing a drug code and a diagnosis code to a drug database system. For example, validation system 102 may provide, to drug database system 108 (e.g., a Drug Utilization Review (DUR) system, such as Medi-Span®, and/or the like, etc.), the drug code (e.g., the NDC, etc.) and the diagnosis code (e.g., the ICD-10 code, etc.). As an example, validation system 102 may transmit, to drug database system 108, the drug code and the diagnosis code. As an example, and referring again to FIG. 4, as shown at reference number 408, validation system 102 may push, via an API connection, the drug code and the diagnosis code to drug database system 108 (e.g., validation system 102 may query the drug database using the NDC and the ICD-10 code, etc.).

As shown in FIG. 3, at step 306, process 300 includes receiving an approved dosage and an approved condition from a drug database system. For example, validation system 102 may, in response to providing the drug code and the diagnosis code to drug database system 108, receive, from drug database system 108, an approved dosage and an approved condition. As an example, and referring again to FIG. 4, as shown at reference number 410, validation system 102 may pull, via the API connection with drug database system 108, the approved dosage and the approved condition associated with the NDC and the ICD-10 code.

An approved condition associated with a drug or medication may include a condition for which the drug or medication has been approved (e.g., for which the drug or medication has received FDA approval, etc.). For example, drugs or medications may be approved to treat certain diagnoses or indications, which are approved within the prescribing information from the manufacturer and stored within DUR engines, such as Medi-Span®, and/or the like. An approved dosage associated with a drug or medication may include a dosage at which the drug or medication has been approved for treating an approved condition (e.g., for which the dosage has received FDS approval, etc.). In such an example, an approved dosage associated with a drug may be dependent on a weight of the patient.

“Off-label” drug use or an “off-label” prescription may refer to prescribing a currently available drug or medication for an indication or condition (e.g., a disease, a symptom, etc.) for which the drug or medication is not approved (e.g., for which the drug has not received FDA approval, etc.). For example, a prescription for a drug including a diagnosis code associated with a different condition than an approved condition for that drug may be referred to as an “off-label” prescription.

As shown in FIG. 3, at step 308, process 300 includes generating a validation decision. For example, validation system 102 may generate, using a machine learning model, a validation decision associated with the prescription by providing as input to the machine learning model the drug code, the diagnosis code, the prescribed dosage, the prescribed quantity, the approved dosage, and/or the approved condition, and receiving as output from the machine learning model, the validation decision. The validation decision may include an indication that the prescription is one of valid and invalid. In some non-limiting embodiments or aspects, validation system 102 may generate, using the machine learning model, the validation decision associated with the prescription by further providing as input to the machine learning model at least one parameter of the patient data associated with the patient (e.g., a patient name, a patient date of birth or age, a patient sex, a patient weight, a patient health condition, a patient allergy, a patient medication, etc.). As an example, and referring again to FIG. 4, at reference number 412, validation system 102 may validate, using the machine learning model, parameters or data points included in the prescription, for example, the NDC, the ICD-10 code, the directions, the quantity, the patient data, etc., against the approved dosage and/or approved condition or indication retrieved from drug database system 108.

Validation system 102 may generate the machine learning model (e.g., an estimator, a classifier, a prediction model, a validation model, etc.) using machine learning techniques including, for example, supervised and/or unsupervised techniques, such as decision trees (e.g., gradient boosted decision trees, random forests, etc.), logistic regressions, artificial neural networks (e.g., convolutional neural networks, etc.), Bayesian statistics, learning automata, Hidden Markov Modeling, linear classifiers, quadratic classifiers, association rule learning, and/or the like. The machine learning model may be trained to provide an output including a validation decision (e.g., an indication of whether a prescription is valid or invalid, etc.) in response to input including parameters associated with the prescription and/or the patient (e.g., the drug code, the diagnosis code, the prescribed dosage, the prescribed quantity, the approved dosage, the approved condition, one or more parameters of the patient data, etc.). For example, validation system 102 may train the model based on training data (e.g., a training dataset, training hashes, etc.) associated with one or more prescriptions for one or more conditions for one or more patients. In such an example, a validation decision may include a probability score associated with the validation decision. For example, the validation decision may include a probability that the prescription is valid (or invalid). In some non-limiting embodiments, validation system 102 may store the model (e.g., store the model for later use). In some non-limiting embodiments or aspects, validation system 102 may store the model in a data structure (e.g., a database, a linked list, a tree, etc.). In some non-limiting embodiments, the data structure is located within validation system 102 or external (e.g., remote from) validation system 102 (e.g., within pharmacy system 106, etc.).

In some non-limiting embodiments or aspects, validation system 102 may automatically generate a validation decision including an indication that a prescription is invalid in response to one or more parameters associated with the prescription and/or the patient being unavailable to validation system 102. For example, if the prescription is for a drug associated with an approved dosage that is dependent on a weight of the patient, and if the patient weight is unavailable within pharmacy system 106 and/or through data supplementation by validation system 102, validation system 102 may automatically (e.g., without executing the machine learning model, etc.), generate a validation decision including an indication that the prescription is invalid to lock processing of the prescription in pharmacy system 106 until the prescription is manually validated by a pharmacist. As an example, if the prescription does not include an NDC code and/or an ICD-10 code, validation system 102 may automatically (e.g., without executing the machine learning model, etc.), generate a validation decision including an indication that the prescription is invalid to lock processing of the prescription in pharmacy system 106 until the prescription is manually validated by a pharmacist.

As shown in FIG. 3, at step 310, process 300 includes providing a validation decision to a pharmacy system. For example, validation system 102 may provide, to pharmacy system 106, the validation decision. The validation decision may cause pharmacy system 106 to one of (i) automatically validate the prescription (and/or enable filling and dispensing of the prescribed medication to a patient, etc.), in response to the validation decision including the indication that the prescription is valid and (ii) automatically lock processing of the prescription such that the pharmacy system 106 is prevented from filling the prescription until manual input associated with the prescription is received from a pharmacist, in response to the validation decision including the indication that the prescription is invalid. As an example, and referring again to FIG. 4, at reference number 414, validation system 102 may control pharmacy system 106 to one of (i) automatically validate the prescription, in response to the validation decision including the indication that the prescription is valid and (ii) automatically lock processing of the prescription such that the pharmacy system 106 is prevented from filling the prescription until manual input associated with the prescription is received from a pharmacist (e.g., a user of pharmacy system 106 with pharmacist level access to the system that is higher than, for example, technician level access, etc.), in response to the validation decision including the indication that the prescription is invalid.

In such an example, if the validation decision includes the indication that the prescription is invalid that causes the pharmacy system 106 to automatically lock processing of the prescription (e.g., prevents a pharmacy technician or other user with lower level pharmacy system access than the pharmacist to fill the prescription, etc.) at reference number 416, pharmacy system 106 may receive, from a pharmacist, manual input that one of (i) confirms the invalidity of the prescription and (ii) overrides the machine generated validation decision based on the clinical knowledge of the pharmacist to create an intervention to prescriber system 104 and validates the prescription for filling (e.g., enables a pharmacy technician or other user with lower level pharmacy system access than the pharmacist to fill the prescription, etc.), thereby automatically and efficiently validating the prescriptions based on the prescribing information to catch errors. If the validation decision includes the indication that the prescription is valid that causes the pharmacy system 106 to automatically validate the prescription, the prescription may immediately become available for filling in pharmacy system 106 by a pharmacy technician or other user with lower level pharmacy system access than the pharmacist, thereby removing the pharmacist from the validation or data verification step of the retail workflow to give back valuable pharmacist time to their patients, their employer, as well as the healthcare system, by counseling patients and providing other services pharmacists were trained to do, for example giving vaccinations.

In some non-limiting embodiments or aspects, providing the validation decision to pharmacy system 106 causes pharmacy system 106 to automatically print a label associated with the prescription. For example, pharmacy system 106 may include a printer configured to print a label that a technician scans when filling a prescription and places on a container including the filled prescription. As an example, validation system 102 may control pharmacy system 106 to automatically print the label for a prescription in response to validating that prescription. The printed label may serve as a notification to the technician that the prescription has been validated for filling.

As shown in FIG. 3, at step 312, process 300 includes receiving an outcome associated with a prescription from a pharmacy system. For example, validation system 102 may receive, from pharmacy system 106, an outcome associated with the prescription. As an example, an outcome associated with a prescription may include a label indicating that the pharmacist confirmed a validation decision indicating that a prescription is invalid (e.g., a true positive label, etc.) or a label indicating that the pharmacist overrode a validation decision indicating that a prescription is invalid (e.g., a false positive label, etc.). For example, and referring again to FIG. 4, at reference number 418, validation system 102 may receive, from pharmacy system 106, an outcome associated with the prescription (e.g., a prescription for which the diagnosis code is associated with a different condition than the approved condition, etc.). In some non-limiting embodiments or aspects, validation system 102 may receive, from pharmacy system 106, a plurality of outcome decisions associated with a plurality of prescriptions at a same time (e.g., in a batch form, etc.).

As shown in FIG. 3, at step 314, process 300 includes updating a validation model. For example, validation system 102 may update, based on the outcome associated with the prescription, the machine learning model. As an example, validation system 102 may train (e.g., retrain, update, etc.) the machine learning model based on the prescription and/or the outcome including the label for the prescription (e.g., based on updated training data, etc.). For example, and referring again to FIG. 4, at reference number 420, validation system 102 may train or update the model based on the updated training data (e.g., using an objective function that depends on the outcome/label, the validation decision, etc.). In such an example, if the prescription is correct but simply written outside of the prescribing information (e.g., off-label, etc.), by using the outcome/label indicating that the pharmacist overrode the validation decision to update or retrain the machine learning model, validation system 102 may learn the nuances of pharmacy practice and off label dosing. In this way, off-label dosing that is overridden enough times may no longer be flagged by validation system 102, which may continue to learn based on the pharmacist's actions. In such an example, validation system 102 may store the updated model (e.g., store the updated model for later use), and/or validation system 102 may continuously train and/or update the machine learning model as new prescriptions and/or outcomes associated therewith are received.

Accordingly, non-limiting embodiments or aspects of the present disclosure may enable precision accuracy when validating prescriptions while also leveraging machine learning algorithms to learn nuances within a pharmacy (e.g., off label dosing, etc.) by providing an automatic and continuously learning validation system for validating prescriptions based on approved prescribing information from the manufacturer and flagging the pharmacist for any contraindications or missing data elements.

### EXAMPLES

A common prescription seen in retail pharmacy is a prescription for antibiotics. Antibiotics are used to treat various infections, but oftentimes there are significantly different dosing of the same medication based on the infection. Knowing a person's weight and diagnosis may be data points that many retail pharmacists do not have access to today. In the hectic retail environment, these missing data elements may be assumed based on clinical experience and/or a quick conversation with the patient or guardian. High dose antibiotics can lead to diarrhea, dehydration, and ultimately an emergency room visit. Non-limiting embodiments or aspects of the present disclosure may capture the medication NDC and ICD-10 diagnosis code in order to drive to a correct indication, therefore, capturing the correct dosing in order to validate the prescription. The quantity and directions may correlate to this dose and the prescription may be validated based on the approved indication. In contrast, existing pharmacy systems do not consider ICD-10 diagnosis codes for validating prescriptions. Many retail pharmacies have current connectivity to DUR engines, such as Medi-Span®. Medi-Span® pulls drug utilization review alerts based only on the NDC and flags the prescription for manual pharmacist intervention and override. These DUR flags in retail pharmacy have become like white noise to the pharmacist and are not diagnosis specific, thus leading to errors. Artificial intelligence does not hear white noise and by pulling in additional patient specific data elements such as ICD-10, comorbidities, allergies, and patient weight, non-limiting embodiments or aspects of the present disclosure may avoid overlooking a contraindication and/or a high dose alert and/or avoid flagging any DUR warnings unnecessarily. Non-limiting embodiments or aspects of the present disclosure may learn based on data or experience from pharmacist intervention analogous to a new pharmacist that calls the prescriber for the majority of the prescriptions that they see on their first day, where a pharmacist with twenty years' experience may know not to call the physician on those same prescriptions. What may be harder to catch, whether it is a pharmacist of one day or a pharmacist with twenty years' experience are prescribing errors. Knowing your patients and having conversations and counseling them often uncovers these errors, and provides another example where non-limiting embodiments or aspects of the present disclosure may bring significant value.

A common mistake shown in pharmacy school is Lamictal versus Lamisil, one is a mood stabilizer and one is an antifungal; this is a common look-a-like sound-alike mistake. Knowing the diagnosis or hard stopping the system if a diagnosis is not on the prescription may catch these mistakes from prescribers. Lamictal, which is a mood stabilizer, with an anti-fungal diagnosis may be flagged and an intervention may be created. In the current landscape of pharmacy, this error may be missed, and has been missed by pharmacists historically. Non-limiting embodiments or aspects of the present disclosure may reduce or prevent these errors by validating diagnosis, medication, and/or directions.

Non-limiting embodiments or aspects of the present disclosure may use various data elements, such as weight, comorbidities, and/or the like to hard stop or lock the pharmacy system and ensure that these data elements are collected to avoid a potential medication error. Data supplementation may aid non-limiting embodiments or aspects in ensuring that not only the correct dose is being prescribed for the patient's weight and diagnosis, but also that the correct medication is being prescribed based on the patient's risk factors or comorbidities. Non-limiting embodiments or aspects may capture how patients performed on a specific medication and if there may be a more appropriate therapy based on other patients with a similar clinical profile, thereby further enhancing the role of the pharmacist.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A system, comprising:
at least one processor programmed and/or configured to:
receive, from a pharmacy system, a prescription associated with a patient, wherein the prescription includes a drug code associated with a drug, a diagnosis code associated with a condition associated with the patient, a prescribed dosage associated with the drug, and a prescribed quantity associated with the drug;
provide, to a drug database system, the drug code and the diagnosis code;
in response to providing the drug code and the diagnosis code to the drug database system, receive, from the drug database system, an approved dosage and an approved condition;
generate, using a machine learning model, a validation decision associated with the prescription by providing as input to the machine learning model the drug code, the diagnosis code, the prescribed dosage, the prescribed quantity, the approved dosage, and the approved condition, and receiving as output from the machine learning model the validation decision, wherein the validation decision includes an indication that the prescription is one of valid and invalid; and
provide, to the pharmacy system, the validation decision, wherein the validation decision causes the pharmacy system to one of (i) automatically validate the prescription, in response to the validation decision including the indication that the prescription is valid and (ii) automatically lock processing of the prescription such that the pharmacy system is prevented from filling the prescription until manual input associated with the prescription is received from a pharmacist, in response to the validation decision including the indication that the prescription is invalid.

2. The system of claim 1, wherein the validation decision includes the indication that the prescription is invalid that causes the pharmacy system to automatically lock processing of the prescription such that the pharmacy system is prevented from filling the prescription until the manual input associated with the prescription is received from the pharmacist, and wherein the at least one processor is further programmed and/or configured to:
receive, from the pharmacy system, an outcome associated with the prescription; and
update, based on the outcome associated with the prescription, the machine learning model.

3. The system of claim 2, wherein the diagnosis code is associated with a different condition than the approved condition.

4. The system of claim 3, wherein the outcome associated with the prescription includes an indication that the manual input from the pharmacist validated the prescription by overriding the validation decision including the indication that the prescription is invalid.

5. The system of claim 1, wherein the prescription includes an electronic prescription, and wherein the electronic prescription is generated by a prescriber system.

6. The system of claim 1, wherein the at least one processor is further programmed and/or configured to:
obtain patient data associated with the patient, wherein the patient data includes at least one of the following parameters: a patient name, a patient date of birth or age, a patient sex, a patient weight, a patient health condition, a patient allergy, a patient medication, a contraindication, a comorbidity, a patient lab value, a patient pharmacogenetics factor, or any combination thereof, wherein the at least one processor generates the validation decision associated with the prescription by further providing as input to the machine learning model at least one parameter of the patient data.

7. The system of claim 6, wherein the approved dosage is dependent on a weight of the patient, and wherein the at least one processor generates the validation decision associated with the prescription by further providing as input to the machine learning model the patient weight.

8. A computer-implemented method, comprising:
receiving, with at least one processor, from a pharmacy system, a prescription associated with a patient, wherein the prescription includes a drug code associated with a drug, a diagnosis code associated with a condition associated with the patient, a prescribed dosage associated with the drug, and a prescribed quantity associated with the drug;
providing, with the at least one processor, to a drug database system, the drug code and the diagnosis code;
in response to providing the drug code and the diagnosis code to the drug database system, receiving, with the at least one processor, from the drug database system, an approved dosage and an approved condition;
generating, with the at least one processor, using a machine learning model, a validation decision associated with the prescription by providing as input to the machine learning model the drug code, the diagnosis code, the prescribed dosage, the prescribed quantity, the approved dosage, and the approved condition, and receiving as output from the machine learning model the validation decision, wherein the validation decision includes an indication that the prescription is one of valid and invalid; and
providing, with the at least one processor, to the pharmacy system, the validation decision, wherein the validation decision causes the pharmacy system to one of (i) automatically validate the prescription, in response to the validation decision including the indication that the prescription is valid and (ii) automatically lock processing of the prescription such that the pharmacy system is prevented from filling the prescription until manual input associated with the prescription is received from a pharmacist, in response to the validation decision including the indication that the prescription is invalid.

9. The computer-implemented method of claim 8, wherein the validation decision includes the indication that the prescription is invalid that causes the pharmacy system to automatically lock processing of the prescription such that the pharmacy system is prevented from filling the prescription until the manual input associated with the prescription is received from the pharmacist, and wherein the method further comprises:
receiving, with the at least one processor, from the pharmacy system, an outcome associated with the prescription; and
updating, with the at least one processor, based on the outcome associated with the prescription, the machine learning model.

10. The computer-implemented method of claim 9, wherein the diagnosis code is associated with a different condition than the approved condition.

11. The computer-implemented method of claim 10, wherein the outcome associated with the prescription includes an indication that the manual input from the pharmacist validated the prescription by overriding the validation decision including the indication that the prescription is invalid.

12. The computer-implemented method of claim 8, wherein the prescription includes an electronic prescription, and wherein the electronic prescription is generated by a prescriber system.

13. The computer-implemented method of claim 8, further comprising:
obtaining, with the at least one processor, patient data associated with the patient, wherein the patient data includes at least one of the following parameters: a patient name, a patient date of birth or age, a patient sex, a patient weight, a patient health condition, a patient allergy, a patient medication, a contraindication, a comorbidity, a patient lab value, a patient pharmacogenetics factor, or any combination thereof, wherein the at least one processor generates the validation decision associated with the prescription by further providing as input to the machine learning model at least one parameter of the patient data.

14. The computer-implemented method of claim 13, wherein the approved dosage is dependent on a weight of the patient, and wherein the at least one processor generates the validation decision associated with the prescription by further providing as input to the machine learning model the patient weight.

15. A computer program product comprising at least one non-transitory computer-readable medium including program instructions that, when executed by at least one processor, cause the at least one processor to:
receive, from a pharmacy system, a prescription associated with a patient, wherein the prescription includes a drug code associated with a drug, a diagnosis code associated with a condition associated with the patient, a prescribed dosage associated with the drug, and a prescribed quantity associated with the drug;
provide, to a drug database system, the drug code and the diagnosis code;
in response to providing the drug code and the diagnosis code to the drug database system, receive, from the drug database system, an approved dosage and an approved condition;
generate, using a machine learning model, a validation decision associated with the prescription by providing as input to the machine learning model the drug code, the diagnosis code, the prescribed dosage, the prescribed quantity, the approved dosage, and the approved condition, and receiving as output from the machine learning model the validation decision, wherein the validation decision includes an indication that the prescription is one of valid and invalid; and
provide, to the pharmacy system, the validation decision, wherein the validation decision causes the pharmacy system to one of (i) automatically validate the prescription, in response to the validation decision including the indication that the prescription is valid and (ii) automatically lock processing of the prescription such that the pharmacy system is prevented from filling the prescription until manual input associated with the prescription is received from a pharmacist, in response to the validation decision including the indication that the prescription is invalid.

16. The computer program product of claim 15, wherein the validation decision includes the indication that the prescription is invalid that causes the pharmacy system to automatically lock processing of the prescription such that the pharmacy system is prevented from filling the prescription until the manual input associated with the prescription is received from the pharmacist, and wherein the program instructions, when executed by the at least one processor, further cause the at least one processor to:
receive, from the pharmacy system, an outcome associated with the prescription; and
update, based on the outcome associated with the prescription, the machine learning model.

17. The computer program product of claim 16, wherein the diagnosis code is associated with a different condition than the approved condition.

18. The computer program product of claim 17, wherein the outcome associated with the prescription includes an indication that the manual input from the pharmacist validated the prescription by overriding the validation decision including the indication that the prescription is invalid.

19. The computer program product of claim 15, wherein the prescription includes an electronic prescription, and wherein the electronic prescription is generated by a prescriber system.

20. The computer program product of claim 15, wherein the program instructions, when executed by the at least one processor, further cause the at least one processor to:
obtain patient data associated with the patient, wherein the patient data includes at least one of the following parameters: a patient name, a patient date of birth or age, a patient sex, a patient weight, a patient health condition, a patient allergy, a patient medication, a contraindication, a comorbidity, a patient lab value, a patient pharmacogenetics factor, or any combination thereof, wherein the at least one processor generates the validation decision associated with the prescription by further providing as input to the machine learning model at least one parameter of the patient data,
wherein the approved dosage is dependent on a weight of the patient, and wherein the at least one processor generates the validation decision associated with the prescription by further providing as input to the machine learning model the patient weight.
